(19) United States
(12) Patent Application Publication
(10) Pub. No.: US 2013/0331683 A1
(43) Pub. Date: Dec. 12, 2013

(54) ELC Thermofilm and Method for Automatically Ascertaining an ELC Thermofilm for Contact Thermography in Order to Detect Temperature Distributions on the Surface, in Particular the Chest, of a Living Being, in Particular a Human Being

(71) Applicant: Heinrich Wehberg, Langwedel (DE)

(72) Inventor: Heinrich Wehberg, Langwedel (DE)

(73) Assignee: SALUTOGENESIS MEDLZINTECHNIK GmbH & Co. KG, Munster (DE)

(21) Appl. No.: 14/001,589

(22) PCT Filed: Feb. 16, 2012
(86) PCT No.: PCT/DE2012/000151

§ 371 Date: Aug. 26, 2013

(30) Foreign Application Priority Data
Feb. 25, 2011 (DE) 10 2011 012 432.

(51) Int. Cl. A61B 5/01

(52) U.S. Cl. CPC A61B 5/015
USPC 600/407

(57) ABSTRACT

An ELC thermal film, in particular for automatically determining an ELC thermal film for contact thermography for detecting temperature distributions on the surface, in particular the breast, of a living organism, in particular of a human being, comprising a carrier film, if appropriate a black layer on the carrier film and a layer containing, preferably microencapsulated, cholesteric liquid crystals on the carrier film or black layer, characterized in that the layer containing the liquid crystals is subdivided in a grid- or strip-shaped fashion into regions having at least two different mixtures of different cholesteric liquid crystals, and a method for automatically determining an ELC thermal film for contact thermography for detecting temperature distributions on the surface, in particular the breast, of a living organism, in particular of a human being

The present application is a National Stage application of International Patent Application Number PCT/DE2012/000151 filed Feb. 16, 2012, which claims priority to German Patent Application 102011012432.2-52, filed Feb. 25, 2011, both of which are incorporated herein by reference.

The present application relates to an ELC thermal film, in particular for automatically determining an ELC thermal film for contact thermography for detecting temperature distributions on the surface, in particular the breast, of a living organism, in particular of a human being, comprising a carrier film, if appropriate a black layer on the carrier film and a layer containing microencapsulated, cholesteric liquid crystals on the carrier film or black layer and to a method for automatically determining an ELC thermal film for contact thermography for detecting temperature distributions on the surface, in particular the breast, of a living organism, in particular of a human being.

ELC (Encapsulated Liquid Crystal) thermography, also called liquid crystal thermography, contact thermography or plate thermography, serves for detecting areal temperature ranges or human body surfaces with the aid of liquid crystals of cholesterol ester, which exhibit characteristic temperature-dependent color changes in respective temperature sensitivity intervals or ranges.

In clinical mammary diagnostics, liquid crystal thermography became widely applicable with the introduction of the thermographic plate for measuring temperature distributions in accordance with DE 25 36 773 C3. In these methods, the microencapsulated liquid crystals are applied onto or into a flexible film, also called ELC thermal film. These ELC thermal films can then be applied directly to the breast or clamped into a rigid frame beforehand. Liquid crystal thermography or plate thermography is a contact-thermal examination method that presupposes direct contact between the surface of the skin and the film. It is only then that the crystals in the film can be heated and a colored thermal image can thus arise. This individual thermal image or the image of a relative temperature distribution arises immediately after the thermal film has been applied to the breast. If contact between the ELC thermal film and the breast is interrupted, the image rapidly disappears within seconds. This process can be reproduced as often as desired and is totally harmless to the patient.

EP 1 171 027 B1 discloses a device and a method for recording a thermo-optical image of the female breast. Three ELC thermal films having different starting temperature sensitivity thresholds, such as 28, 30 and 32° C., for example, but uniform temperature sensitivity ranges of 4° C., for example, are often available for the recording of thermo-optical images. Thermal films comprising ELC material are commercially available in a temperature range of 15° C.-50° C. The ELC thermal films are black up to an individual starting temperature sensitivity threshold and then begin to change color, e.g. from red through yellow-green-blue to blue-black again, as the temperature rises. The color spectrum can even be traversed repeatedly, such as two or three times, for example, within the temperature sensitivity range.

For the actual recording, a suitable ELC thermal film has to be selected beforehand from, for example, the three ELC thermal films mentioned above. It is intended to represent the heating process in a highest possible perceptible (measurable) color spectrum. At the present time, the suitable ELC thermal film is determined or selected by a physician or an appropriately trained person as follows: the ELC thermal film having the average starting temperature sensitivity threshold, regularly the ELC thermal film having the starting temperature sensitivity threshold at 30° C., is the first to be placed onto the breast or some other body part to be examined. The other (two) ELC thermal films then follow in order to be able to compare the evolution of heat for the respective ELC thermal films. The ELC thermal film that comes closest to achieving the aim mentioned above is selected simply by observation by eye according to the estimation of said physician/person and with requisite experience. However, if an ELC thermal film having an excessively low starting temperature sensitivity threshold is selected, the result is that the color scale or the color spectrum is not traversed completely but rather for the most part skipped (becomes imperceptible) until reaching the upper end of the temperature sensitivity range.

By contrast, if the ELC thermal film is chosen with an excessively high starting temperature sensitivity threshold, then the result is that only part of the possible color scale is represented and utilized. The color spectrum and thus temperature spectrum present does not become completely perceptible in terms of its differentiation possibility.

Hitherto, films having temperature overlap to a great extent—relative to the respective neighboring films—have been dispensed with, since it is then no longer possible to decide on the correct film with the naked eye.

The invention therefore addresses the problem of enabling a more accurate determination in a preferably automated form of the respective suitable ELC thermal film.

This problem is solved according to the invention by means of an ELC thermal film for contact thermography for detecting temperature distributions on the surface, in particular the breast, of a living organism, in particular of a human being, comprising a carrier film, if appropriate a black layer on the carrier film and a layer containing, preferably microencapsulated, cholesteric liquid crystals on the carrier film or black layer, characterized in that the layer containing the liquid crystals is subdivided in a grid- or strip-shaped fashion into regions having at least two different mixtures of different cholesteric liquid crystals. The ELC thermal film can also be designated as combined film or test film. In this case, the different mixtures are associated with different starting temperature sensitivity thresholds.

Furthermore, this problem is solved by means of a method for automatically determining an ELC thermal film for contact thermography for detecting temperature distributions on the surface, in particular the breast, of a living organism, in particular of a human being, comprising:
applying a thermal film as claimed in any of the preceding claims to the surface of a living organism to be examined and, if appropriate, cooling the thermal film,
recording an image sequence of the heating of the thermal film by means of a, preferably digital, camera over a previously defined recording duration and with a previously defined image recording frequency, image resolution and color depth,
decomposing the image sequence into a number of sub-image sequences corresponding to the number of regions having different mixtures of different cholesteric liquid crystals by, for each image of the image sequence, extracting image raster elements associated with regions having the same mixture of different cholesteric liquid crystals in the thermal film, and joining together same to form a sub-image and by preferably chronologically arranging same,
calculating the sum of the changes in color over time for all pixels in the respective sub-image sequence,
determining the sub-image sequence having the highest sum or two sub-image sequences having similarly high or highest sums, and
preferably automatically outputting the mixture of different cholesteric liquid crystals that is associated with the sub-image sequence having the highest sum or an identifier of the associated starting temperature sensitivity threshold or the mixtures associated with the sub-image sequences having similarly high or highest sums or identifiers of the associated starting temperature sensitivity thresholds or a starting temperature sensitivity threshold between the two associated starting temperature sensitivity thresholds. It goes without saying that, for recording the image sequence, by way of example, an analog video camera could also be used and the image sequence could be digitized subsequently.

In order words, the image color variation dynamic range or pixel color variation dynamic range is evaluated. The color variation of an individual pixel thus has a higher pixel variation dynamic range relative to a fixed color profile when traversing the same number of colors of the color profile in a shorter time. Said dynamic range can be expressed for example in the number of colors traversed per time. Accordingly, an image which consists of many pixels has a higher image color variation dynamic range if the sum of the pixel color variation dynamic range is greater than in a comparative image.

Furthermore, this problem is solved by means of a method for determining an ELC thermal film for contact thermography for detecting temperature distributions on the surface, in particular the breast, of a living organism, in particular of a human being, comprising: successively for at least two thermal films having layers having different mixtures of different, preferably microencapsulated, cholesteric liquid crystals, applying a thermal film having a layer containing exactly one mixture of different, preferably microencapsulated, cholesteric liquid crystals to the surface of a living organism to be examined and, if appropriate, cooling the thermal film, recording an image sequence of the heating of the thermal film by means of a, preferably digital, camera over a previously defined recording duration and with a previously defined image recording frequency, image resolution and color depth, and calculating the sum of the changes in color over time for all pixels in the image sequence, and determining the image sequence having the highest sum or two image sequences having similarly high or highest sums, and preferably automatically outputting the mixture of different cholesteric liquid crystals that is associated with the image sequence having the highest sum or an identifier of the associated starting temperature sensitivity threshold or the mixtures associated with the two image sequences having similarly high or highest sums or identifiers of the associated starting temperature sensitivity thresholds or a starting temperature sensitivity threshold between the two associated starting temperature sensitivity thresholds.

Advantageously, in the case of the ELC thermal film, a protective layer is provided on the layer containing cholesteric liquid crystals.

In the case of the ELC thermal film, the regions are expediently distributed uniformly.

Advantageously, the regions are of the same size. In principle, the regions should be as small as possible. Regions having minimum diameters of the order of magnitude of 1 to 2 mm can be realized at the present time.

In accordance with a further particular embodiment of the invention, the regions are square or rectangular. It goes without saying, however, that other shapes are also possible.

Advantageously, the different mixtures have identical temperature sensitivity ranges. In particular, provision can be made here for at least two neighboring temperature sensitivity ranges to adjoin one another. Alternatively or additionally, provision can be made for at least two neighboring temperature sensitivity ranges to overlap one another.

In the case of the methods, the image sequence is advantageously recorded in the RAW format, other formats also being conceivable.

Advantageously, the recording duration is in the range of approximately two to four seconds.

Expediently, the image recording frequency is greater than or equal to 2 Hz.

Expediently, the image resolution is in the VGA range (640×840 pixels) or higher.

Expediently, the color depth is at least 8 bits.

Advantageously, the color of the pixels is recorded or represented in the RGB (red, green, blue) space.

The invention is based on the surprising insight that by evaluating the pixel color variation dynamic range or the image color variation dynamic range, it is possible to determine a suitable ELC thermal film according to objective criteria in a standardized manner, reproducibly and preferably in automated form. By utilizing one film, the cooling influence by the film itself is always the same. Whereas the undesirable cooling influences always present hitherto owing to the utilization of a plurality of different films precluded a reproducible process. If the combined film is used in accordance with one particular embodiment, then it is even possible also to save time. Hitherto, an image sequence has always been interpreted by the human eye. Interpretability was not possible in the case of thermal films that heat up too rapidly, since the human eye cannot achieve such a high spatial and temporal resolution and cannot process the amount of information. The temperature intervals had to have a minimum size for the eye in order to be able to differentiate them with the naked eye, and were not permitted to overlap, or were permitted to overlap only slightly.

Further features and advantages of the invention are evident from the accompanying claims and the following description which elucidates exemplary embodiments in detail with reference to the schematic drawings, in which:

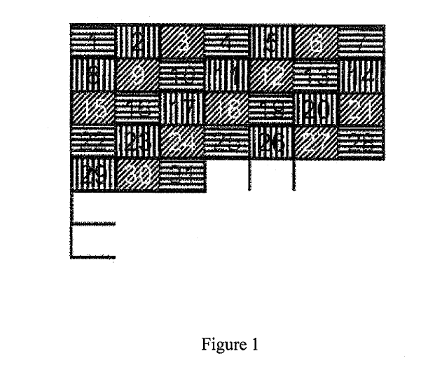

FIG. 1 shows the grid-shaped subdivision of an ELC thermal film in accordance with one particular embodiment of the present invention;

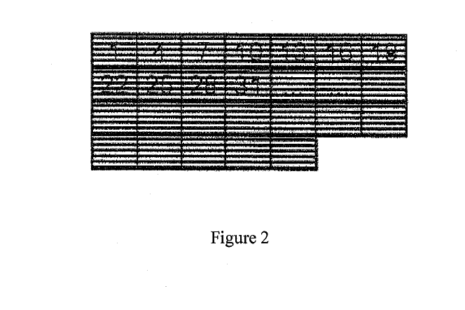

FIG. 2 shows a sub-image of an image sequence for a specific point in time that is based on regions of the ELC thermal film having a low starting temperature sensitivity threshold;

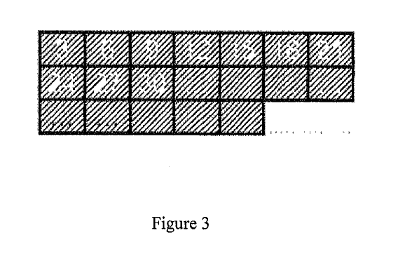

FIG. 3 shows a sub-image of an image sequence for a specific point in time that is based on regions of the ELC thermal film having a medium starting temperature sensitivity threshold;

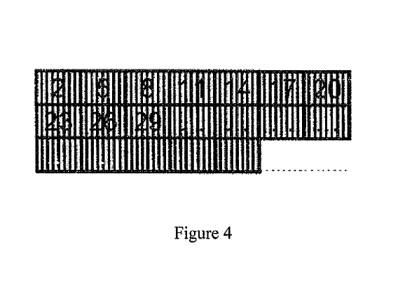

FIG. 4 shows a sub-image of an image sequence for a specific point in time that is based on regions of the ELC thermal film having a high starting temperature sensitivity threshold;

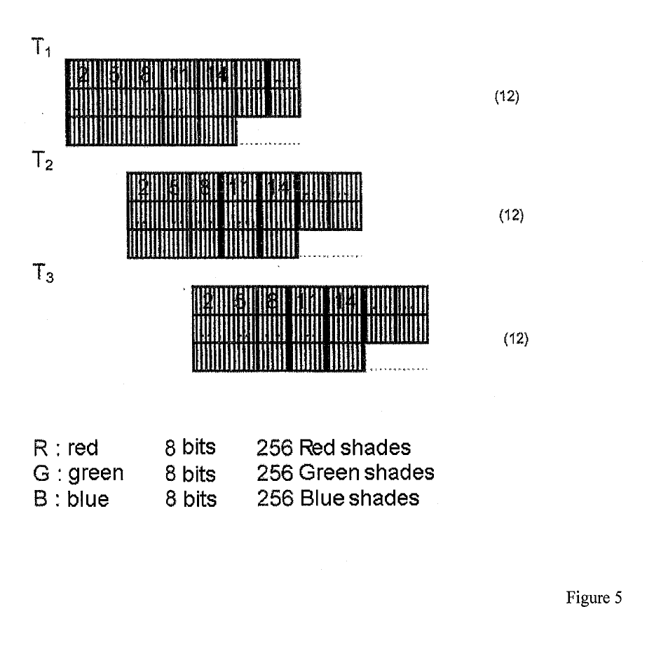

FIG. 5 shows an excerpt from a sub-image that is based on regions of the ELC thermal film having a medium starting temperature sensitivity threshold, at three different points in time of an image sequence; and

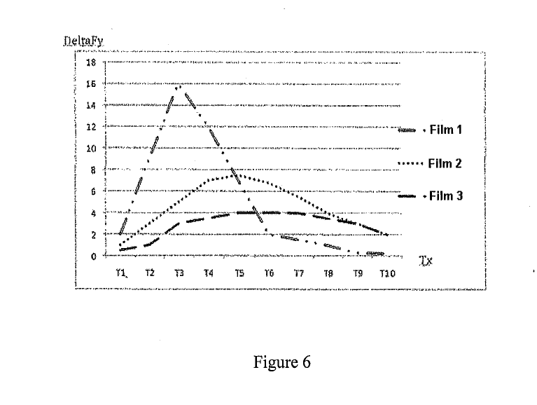

FIG. 6 shows a graphical representation of the sum of the change in color over time for all pixels of the images based on the three different starting temperature sensitivity thresholds, as a function of time.

As is evident from FIG. 1, an ELC thermal film 10 in accordance with one particular embodiment of the present invention is subdivided in a grid-shaped fashion into square regions (approximately 1 cm×1 cm) having three different mixtures of different cholesteric liquid crystals and thus having different starting temperature sensitivity thresholds. The regions having the low starting temperature sensitivity threshold, namely 28° C., are those with the numerals 1, 4, 7, 10, 13, 16, 19, 22, 25, 28, 31, etc. The regions having a medium starting temperature sensitivity threshold of 30° C. are numbered consecutively as follows: 3, 6, 9, 12, 15, 18, 21, 24, 27, 30, etc. Finally, the regions having a high starting temperature sensitivity threshold, namely 32° C., are provided with the numbers 2, 5, 8, 11, 14, 17, 20, 23, 26, 29, etc. It goes without saying that the regions having different starting temperature sensitivity thresholds could also be arranged in a different order and/or a different number of mixtures could be used. Said regions have not only different starting temperature sensitivity thresholds but also temperature sensitivity ranges, which were chosen to be identical in the present case and are approximately 3° C. In order to determine which starting temperature sensitivity threshold is best suited to the most differentiated thermal image, the ELC thermal film 10, also called test film or combined film, is placed for example onto a female breast and, after possible cooling of the ELC thermal film until the liquid crystals no longer reflect, an image sequence of the heating of the ELC thermal film 10 is recorded by means of a digital camera in the RAW format or other formats. In the present example, the image resolution is 640×480 pixels (VGA) with a color depth of 8 bits and an image recording frequency of 2 Hz or more. A respectively higher image resolution and color depth and/or a higher image recording frequency improve the expressive accuracy. The maximum resolution of the ELC thermal film therefore represents a limit for a meaningful image resolution. The spatial resolution of the ELC thermal film goes down to approximately 0.01 mm. The thermal resolution is approximately 0.007° C. in the case of highly sensitive ELC thermal films. Assuming that the temperature sensitivity range is traversed once in 2 seconds, an optimum image recording frequency of 45 Hz results. In the case of homogeneous traversal twice in the same time, an optimum image recording frequency of 90 Hz results.

In one preferred embodiment, the individual digital images are created with an image resolution of 1600×1200 pixels and with an image depth of 24 bits (8 bits each for the colors red, green and blue). A color depth of 48 bits is also possible. The image recording frequency is 90 Hz in the specific embodiment.

The software-aided evaluation of the ELC thermal film 10 comprises extracting image raster elements associated with regions having the same mixture of different cholesteric liquid crystals of the thermal film and joining together the same to form a sub-image 12, as is illustrated in FIGS. 2 to 4 for the regions having a low, medium and high starting temperature sensitivity threshold. Afterward, three sub-image sequences, namely for each starting temperature sensitivity threshold, are created by chronologically arranging the sub-images, as illustrated in FIG. 5 for, by way of example, only three successive points in time for the regions having the higher starting temperature sensitivity threshold. In other words, the image sequence is decomposed into three sub-image sequences. Squares without color variation, for example because no body part is situated there, are not taken into account and are omitted.

From these sub-images created virtually in each case at identical points in time and from sub-image sequences resulting therefrom, a color change progression is determined for each pixel. Said progression is presented below for non-representative color components of an image pixel:
Tx: Point in time of the heating of the ELC thermal film
DeltaFy: Image color variation dynamic range (sum of the positive difference between the three color components (R, G, B))
Film 1 T₁: Red=32, Green=72; Blue=168
Film 2 T₂: Red =35, Green=71, Blue=178
DeltaF₁ T₂−T₁: 3 (Red)+1 (Green)+10 (Blue)=14
Film 1 T₃: Red=38, Green=77, Blue=188
DeltaF₁ T₃−T₂: 3 (Red)+6 (Green)+10 (Blue)=19

Film 1 T₄: Red=50, Green=62, Blue=150

DeltaF+T₄−T₃: 12 (Red)+15 (Green)+38 (Blue)=65

Film 1 T₅: —

DeltaF₁ T₅−T₄: —

FIG. 6 then shows by way of example, nor with reference to the abovementioned values, DeltaF_{y} as a function of the time Tx for three ELC thermal films having different starting temperature sensitivity thresholds. As the starting temperature sensitivity threshold increases (film 1→film 2→film 3→) the curves become flatter and flatter. The sum of the image color variation dynamic ranges DeltaF_{y} Tx over all points in time Tx over the heating interval corresponds to the area beneath the respective curves. The ELC thermal film to be determined is intended to represent the heating process in a highest possible color spectrum, wherein the color spectrum is generally traversed at least twice. The curve with the largest area beneath it belongs to the desired ELC thermal film.

For the case of two approximately identical areas beneath the curves, in other words for the case of similar sums, wherein the similarity can be defined for example by a tolerance threshold of approximately 15 or 20%, an ELC thermal film is to be used whose temperature sensitivity range lies between the temperature sensitivity ranges of the two associated ELC thermal films having similar sums and overlaps the temperature sensitivity ranges that are adjacent to one another, preferably by half in each case.

The assignment of the temperatures corresponding to the colors of the ELC thermal film and the resultant digital colors, which are described with a color depth of 8 bits for the colors blue, red and green, is carried out by means of the following procedure:

The variation of the temperature is evident from the assignment table above. The associated digital color changes, defined by red, green and blue, therefore indicate the temperature of the ELC thermal film at the respective locations. The variation of a hundredth of a degree Celsius is thus defined as one step. The dynamic temperature variation is thus described by the steps per time and thereby becomes measurable and countable.

Another example of the determination of an expedient image resolution and color depth depending on the physical properties of an ELC thermal film (starting point of the salutography) is introduced below.

Physical Properties of ELC Thermal Films:

The temperature resolution capability of a thermal film extends as far as 0.007° C. (Liquid Crystals Ind. Inc., Turtle Creek, Pa. USA —Thermometric Objects or Thermometers and Methods for producing same. DT-OS 2 012 493, Mar. 17, 1970, Union Priority Mar. 13, 1969).

The spatial resolution of a crystalline thermal film is determined as 0.01×0.01 mm.

(WAGNER; K. and H. Liebig: Flussigkristalle—Ihre Anwendung and deren Problematik [Liquid Crystals—their use and problems relating thereto]. Chemiker zeitung 95, 733-736, (1971)).

Determining the Expedient Resolution:

1.) Spatial Resolution:

The required area of the thermal film measures: 25 cm×30 cm.

Pixels can be differentiated per centimeter.

1 cm=100 mm/0.01 mm: 10 000

10 000×25 cm and 30 cm: =250 000×300 000 pixels=75 billion.

2.) The thermal resolution is represented by color differentiation. The differentiability of different hues/color shades, taking account of the speed of a complete color pass, predetermines the image frequency and the required color differentiation possibility (color depth).

In salutography a complete color pass is achieved at the lower end (rapidly) already within a time period of one second. In this case, a color pass has a temperature spectrum of 1.5° C.

1.5° C./0.007° C.=214.5

That results in 214.5 different colors in 1.5 seconds or 142.9 different hues per second.

This results in a required number of image refreshes per second of 142.9 μm in order to ensure no loss.

### SUMMARY

The spatial resolution cannot yet be fully utilized by a marketable device (of interest in terms of price). In this case, however, substantiated on the basis of the already good experience with a resolution of 640×480, a resolution of full HD, approximately 2 million pixels (approximately 8 times VGA 640×480, approximately 3 million pixels), should suffice.

8 bits already suffice for the color depth. Expediently, 24 bits are employed here in order to reliably preclude a loss at this point with the existing standard.

For the image refresh, as already for the spatial resolution, the highest possible frequency should be chosen since the optimum value of a frequency of 143 will not be expedient owing to the price-performance standpoint. In digital photography, image frequencies of up to 60 Hz as standard are offered for FULL HD.

The features of the invention disclosed in the present description, in the drawings and in the claims may be essential to the realization of the invention in its various embodiments both individually and in any desired combinations.

### LIST OF REFERENCE SIGNS

10 ELC thermal film
12 Sub-image

1. An ELC thermal film (10), in particular for automatically determining an ELC thermal film (10) for contact thermography for detecting temperature distributions on the surface, in particular the breast, of a living organism, in particular of a human being, comprising a carrier film, if appropriate a black layer on the carrier film and a layer containing, preferably microencapsulated, cholesteric liquid crystals on the carrier film or black layer, characterized in that
the layer containing the liquid crystals is subdivided in a grid- or strip-shaped fashion into regions having at least two different mixtures of different cholesteric liquid crystals.

2. The thermal film as claimed in claim 1, characterized in that a protective layer is provided on the layer containing cholesteric liquid crystals.

3. The thermal film as claimed in claim 1, characterized in that the regions are distributed uniformly.

4. The thermal film as claimed in claim 1, characterized in that the regions are of the same size.

5. The thermal film as claimed in claim 1, characterized in that the regions are square or rectangular.

6. The thermal film as claimed in claim 1, characterized in that the different mixtures have identical temperature sensitivity ranges.

7. The thermal film as claimed in claim 6, characterized in that at least two neighboring temperature sensitivity ranges adjoin one another.

8. The thermal film as claimed in claim 6, characterized in that at least two neighboring temperature sensitivity ranges overlap one another.

9. A method for automatically determining an ELC thermal film (10) for contact thermography for detecting temperature distributions on the surface, in particular the breast, of a living organism, in particular of a human being, comprising:
applying a thermal film to the surface of a living organism to be examined and, if appropriate, cooling the thermal film,
recording an image sequence of the heating of the thermal film by means of a, preferably digital, camera over a previously defined recording duration and with a previously defined image recording frequency, image resolution and color depth,
decomposing the image sequence into a number of sub-image sequences corresponding to the number of regions having different mixtures of different cholesteric liquid crystals by, for each image of the image sequence, extracting image raster elements associated with regions having the same mixture of different cholesteric liquid crystals in the thermal film, and joining together same to form a sub-image (12) and by chronologically arranging same,
calculating the sum of the changes in color over time for all pixels in the respective sub-image sequence,
determining the sub-image sequence having the highest sum or two sub-image sequences having similarly high or highest sums, and
preferably automatically outputting the mixture of different cholesteric liquid crystals that is associated with the sub-image sequence having the highest sum or an identifier of the associated starting temperature sensitivity threshold or the mixtures associated with the two sub-image sequences having similarly high or highest sums or identifiers of the associated starting temperature sensitivity thresholds or a starting temperature sensitivity threshold between the two associated starting temperature sensitivity thresholds.

10. A method for determining an ELC thermal film (10) for contact thermography for detecting temperature distributions on the surface, in particular the breast, of a living organism, in particular of a human being, comprising:
successively for at least two thermal films having layers having different mixtures of different, preferably microencapsulated, cholesteric liquid crystals
applying a thermal film having a layer containing exactly one mixture of different, preferably microencapsulated, cholesteric liquid crystals to the surface of a living organism to be examined and, if appropriate, cooling the thermal film,
recording an image sequence of the heating of the thermal film by means of a, preferably digital, camera over a previously defined recording duration and with a previously defined image recording frequency, image resolution and color depth, and
calculating the sum of the changes in color over time for all pixels in the image sequence, and
determining the image sequence having the highest sum or two image sequences having similarly high or highest sums, and
preferably automatically outputting the mixture of different cholesteric liquid crystals that is associated with the image sequence having the highest sum or an identifier of the associated starting temperature sensitivity threshold or the mixtures associated with the two image sequences having similarly high or highest sums or identifiers of the associated starting temperature sensitivity thresholds or a starting temperature sensitivity threshold between the two associated starting temperature sensitivity thresholds.

11. The method as claimed in claim 9, characterized in that the image sequence is recorded in the RAW format.

12. The method as claimed in claim 9, characterized in that the recording duration is in the range of approximately two to four seconds.

13. The method as claimed in claim 9, characterized in that the image recording frequency is greater than 2 Hz.

14. The method as claimed in claim 9, characterized in that the image resolution is in the VGA range or higher.

15. The method as claimed in claim 9, characterized in that the color depth is at least 8 bits.

16. The method as claimed in claim 9, characterized in that the color of the pixels is recorded or represented in the RGB space.
